(19) United States
(12) Patent Application Publication
(10) Pub. No.: US 2005/0039744 A1
(43) Pub. Date: Feb. 24, 2005

(54) Delivery of dispersed powders

(76) Inventor: Stephen George Szirmai, New South Wales (AU)

Correspondence Address: LADAS & PARRY, 5670 WILSHIRE BOULEVARD, SUITE 2100, LOS ANGELES, CA, 90036-5679, US

(21) Appl. No.: 10/501,661

(22) PCT Filed: Nov. 15, 2003
(86) PCT No.: PCT/AU03/01521

(30) Foreign Application Priority Data
Nov. 15, 2002 (AU) 2002952683

(51) Int. Cl. A61M 15/00; A61M 16/00

(52) U.S. Cl. 128/203.15; 128/203.12

(57) ABSTRACT

Apparatus for fine powder dissension, such as dispensing micronised pharmaceutical powder during inhalation therapy, has a container (19) for receiving the fine powder and beads (typically of millimetre size) with a high speed rotor (15) adapted to receive electrostatic charge and through sharp metal arms (17) to establish an electric field while the arm (17) mechanically operates to develop a cloud of fine particles which is discharged through an outlet mesh (21) which retains the beads in the container.

### FIELD OF THE INVENTION

The present invention relates to the delivery of dispersed powders and manifests itself in terms of novel apparatus, novel methods and novel parts and kits which in use generate solid aerosols.

The present invention will be particularly described with reference to generating solid aerosols intended for the inhaled delivery of dry substances to the respiratory tract. However, the invention is not necessarily confined only to such applications and is to be considered applicable to any equivalent applications.

### BACKGROUND OF THE INVENTION

The present invention uses and is based on the known concept of electro-suspension. This is an electrostatic effect previously reported by Stephen G Szirmai in the Journal of Applied Physics, 1980, 51 (10) 5215-5222 and 5223-5227, and in the Journal of Applied Physics 1984, 55 (11) 4088-4094. As reported, the electrosuspension effect allows the suspension of dry powders within closed or open containers purely through the application of an intense DC voltage gradient. This causes the formation of a suspended cloud of dust above, and in dynamic equilibrium with, the rest of the static powder bed.

It was further disclosed that many powders, e.g. ultrafine or micronised pharmaceutical substances, can not be dispersed by simply applying an intense electrostatic field across the bed, but require a special electrode geometry which ensures that the applied intense electrostatic field is also combined with air-ionisation in order to charge the particles. These, as well as practical examples were discussed in U.S. Pat. No. 5,463,524 (Szirmai) and corresponding European patent application 90911890.3. These specifications also disclose the possible use of electrostatic powder suspension (‘electrosuspension’) for delivering powdered anti-asthmatic agents to the respiratory tract.

Currently, devices for delivering medical aerosols to the respiratory tract include the following broad categories:
(1) metered dose inhalers,
(2) dry powder inhalers
(3) nebulisers

Of these, only (1) and (2) deliver dry powders, so that nebulisers, (which are based on ultrasonic as well as other atomisation techniques for dispersing liquid solutions into droplet form), are not relevant to the field of the present invention.

There are a number of different device designs for personal dry-powder delivery in the treatment of asthmatic conditions, most are based on one of two operating principles broadly in line with the above categories. The first category comprises metered dose inhalers which use a liquefied solvent gas to carry the active drug as well as to act as a propellent. When a dose is released into the mouth, the solvent evaporates so that the substance is delivered as a dry powder. However, metered dose inhalers suffer from a number of practical shortcomings (see ‘Asthma Inhalation Therapy’, Med. Sci. Bull. 1996, 19(3), 1), including the problem that a patient is unable directly to tell how much medication is left inside the pressurised canisters. The only suggested technique is to float a canister on water and determine the contents on a ‘sink or float’ basis. However, recently this practice has been criticised by Glaxo Wellcome, the makers of one type of such device known as ‘Ventolin Inhaler’. Another problem with metered dose inhalers is that on activating such a device, it delivers a sudden burst of the contents, requiring patients to coordinate their breathing with the delivery. This is especially troublesome for children who must practice their breathing for this purpose. Finally, metered dose inhalers are often criticised on the basis of being environmentally harmful, due to the propellent gas which is released to the atmosphere.

The second broad category (dry powder inhalers) comprises inhalers based on breath activation, where the powder is inhaled by the patient by using the vacuum generated by the act of inhaling to create the dry powder aerosol. While this method overcomes problems of coordination and has no environmentally damaging by-products, experience with these devices (e.g. such as those known as Rotohaler, Diskhaler and Turbohaler) is that heavy asthmatics cannot generate sufficient suction to obtain a full dosage. Due to their reduced lung capacity, the same problem exists for small children. In addition, breath activated devices also require a large quantity of neutral filler such as lactose, usually providing over 95% of the material, mixed with the active substance in order to aid physically the delivery process. The lactose, while chemically neutral, is known to cause allergic reactions in some individuals.

The above devices are generally unable to deliver more than 15%-20% of the active substance to the lungs, with the rest of the inhaled dust being trapped by the mucous membranes of the mouth cavity and thus being swallowed.

Electrosuspension powder delivery, a technique utilised as an element of the present invention, does not fit into the two categories described above, since a cloud of particulates is generated and propelled by electrostatic forces; propellent gases or solvents are not used and neither is breath activation required. However, the apparatus, as suggested in U.S. Pat. No. 5,463,524 (Szirmai) and equivalents, has not found favour for the purpose of therapeutic use. Accordingly, more attractive delivery systems using inherent characteristics of electrosuspension techniques would be desirable.

### SUMMARY OF THE INVENTION

In a first aspect the invention consists of an apparatus for dispersing a fine powder, the apparatus including a container for receiving contents which include the fine powder and beads of a relatively large dimension compared with the powder, a rotor rotatably mounted within the container for co-dispersing the contents when the rotor is driven, means for rotating the rotor at relative high speed, a first electrode and a second electrode connected to means for applying an electric field in the container, a discharge port from the container having means to permit discharge of a dispersion of fine powder from within the container and means to retain the beads within the container.

In a further aspect, the invention consists in a method for dispersing a fine powder comprising using an apparatus as described in the first aspect above.

The method may also be expressed as a method of inhalation therapy wherein the method includes patient activation of a drive system for the rotor and inhaling the dispersion of fine powder from the discharge port.

The invention also extends to sub-combinations and components for the apparatus and dosage units of fine powders specifically packaged and configured for loading and use in the apparatus and method according to the above described aspects.

In contrast to the prior art described hereinbefore, the present invention makes use of a combination of factors which may be described as comprising:
(a) the use of electromechanical forces due to the rotor;
(b) the effect of the beads within the container; and
(c) the combination with electrostatic forces which form an electrosuspension of the fine powder.

It is believed that the concept of using the electromechanical forces and the supply of beads within the container deals effectively with a problem the present inventor has appreciated as inherent in the prior art and specifically in U.S. Pat. No. 5,463,524 namely that there is believed to be a rapid build-up of electrically charged fine powder on the internal walls of the equipment with consequent reduction of the emitted dose levels. When the dose repeatability is measured using scatter statistics for RSD (relative standard deviation), one finds that this can be as high as 80% or more, while the industry standard requires RSD to be generally below 8%. By contrast embodiments of the present invention can overcome this severe disadvantage with the prior art.

Another requirement which cannot be met with the prior art device relates to the respirable fraction of the emitted dust; this fraction must be above 30% in order to represent a useful advance over present technologies. It has been found that embodiments of the invention can achieve those requirements.

A further technical difficulty in the prior art is the requirement for a high internal electrostatic field, often requiring DC voltages in excess of 12 kV. This places serious restrictions on the portability of such a device as it has to be connected to a high voltage power source. Embodiments of the present invention can be highly portable, and may be powered with a small battery driven motor.

Yet a further problem experienced with methods according to the prior art patents is that rotor design only permits speeds that are generally below 3,000 rpm, as higher velocity will stress a thin corona-wire construction, causing the eventual disintegration of the rotor. A further inherent limitation with prior art equipment is that a considerable time-delay exists between switching on the device and producing the powder suspension, a time-lag unacceptably high for inhaled dosage delivery which has a typical time span of 2-3 seconds. At least preferred embodiments of the invention overcome these problems.

By contrast to the prior art device, it is believed that the embodiments of the present invention can offer numerous practical and performance advantages outlined in more detail below and which offer a compact, portable and reliable device capable of repeated dosage for patient inhalation to maintain the powder in a form which causes a predictably high proportion of the active ingredient to reach the target zone in the lungs and in quantities which, dose after dose, are consistently the same.

Accordingly it is believed that embodiments of the invention open up the potential for easy inhalation therapy and can readily be used by young children and weak patients as well as asthmatics.

A wide range of powders with different characteristics can be accommodated; many pharmaceutical powders have been found to be inherently difficult to disperse and to maintain in an effective dispersement. Agglomeration of micronised powders is to be avoided as agglomeration tends to prevent powders reaching the target tissues in the lungs and instead are taken up by mucous of the body. Thus the powders are ingested and are not effective for the purpose intended.

Furthermore, as a consequence of maintaining the desired dispersion, it is believed a high degree of accuracy of dosage into the target tissues of the patient's lungs can be achieved. Therefore pharmaceuticals likely to be dangerous if inhaled in excessive doses can be considered for inhalation therapy.

Preferred embodiments may adopt further inventive features which in general can be adopted in any combination or permutation and which will now be described.

The rotor can function as the first electrode and may provide a multiplicity of blade-like elements spaced from the axis of rotation and from one another. Each blade element can have a surface extending substantially redially from the axis of rotation, to provide electrical interaction with the contents of the container.

The drive means can be arranged to drive the rotor at high speed, e.g. of the order of 9,000 rpm.

The design of the rotor and its drive system, with advantage, can be such as to utilise the Van de Graff effect to generate an electrostatic field of the order of 12 kV.

Embodiments of the invention also can utilise an outlet screen having an array of apertures to permit the discharge and dispersion of powder of very fine dimension only e.g. less than 10 microns, thereby retaining not only the beads but also any agglomerations of particles or coarser particles.

The form of the rotor is such that the beads impact on the output screen, tending to break up and remove entrained coarser particles and agglomerated particles whereby they are in general then susceptible to being taken up in the electrodispersion to be discharged.

A particularly useful form of this inventive aspect is when the outlet is a double mesh, the meshes being separated by 0.1 to 1 mm. The inner mesh is coarser and the outer mesh is fine so that coarser particles and agglomerated particles are entrained between the mesh and under impact of the beads this permits fine particles to fall down into the container for subsequent electrodispersion. The outer mesh typically has a transverse opening space of about 10 microns and the lower mesh typically about 75 microns.

Although a variety of materials can be chosen, the inventors have found that silicon rubber beads work effectively and these beads are believed to clean the inner surfaces of the apparatus including the rotor and the underside of the outlet screen thereby preventing the build up of charged particles on these surfaces.

Another advantageous feature which may be incorporated in at least some embodiments is the provision of additional fine powder between the two layers of a double screen. This material may comprise a supply of micronised pharmaceutical drug powder together with or substituted by a substantially inert substance such as alumina powder.

With advantage the invention can be implemented in a compact device in which drive to the rotor of the device is provided by e.g. two low voltage cells. For example two AA size 1.5 volt batteries have been found effective to drive the rotor system which can generate up to 12 kV of electrostatic field.

It is believed that where the preferred embodiment is employed with sharp blades provided to act as a corona ionisation source, the design facilitates an inherent high degree of mechanical strength in the rotor permitting high speed operation up to 9,000 rpm, this rotor contributing the operation of the device in providing electromechanical forces to aid operation of the device.

It has been found convenient and effective to use spherical beads in the device but other shapes may be useable. The number of beads used and their size are arbitrary; in practice it has been found useful to use beads of a few millimetres diameter, say 2 to 3 mm and in the device to have between 50 and 100 beads.

The inventors envisage that a variety of shapes and configurations of the device can be developed to embody the invention. For example a split system architecture can be devised with the motor drive/generators and battery compartment arranged to be retained by the user while the portion of the device which encloses the dispersing volume, the rotor and screen assembly would be mountable. This allows this portion to be discarded after it has exhausted the drugs supplied in the component. This may also permit a patient to have a multiplicity of drugs to be inhaled at different times and the patient only has to purchase and carry a single base unit.

For particular applications various optional features may be required and can be readily incorporated into embodiments of the invention. For example an electronic timer can be incorporated to facilitate automatic dose control and indeed interval between doses. An inhalation tube for the patient could also be provided.

### BRIEF DESCRIPTION OF THE DRAWINGS

For illustrative purposes only an embodiment of the invention will now be described with reference to the accompanying drawing which is a cut-away isometric view illustrating schematically an embodiment of the invention and being of the nature of a laboratory prototype for delivering dry aerosols to the respiratory tract.

### DETAILED DESCRIPTION OF THE DRAWINGS

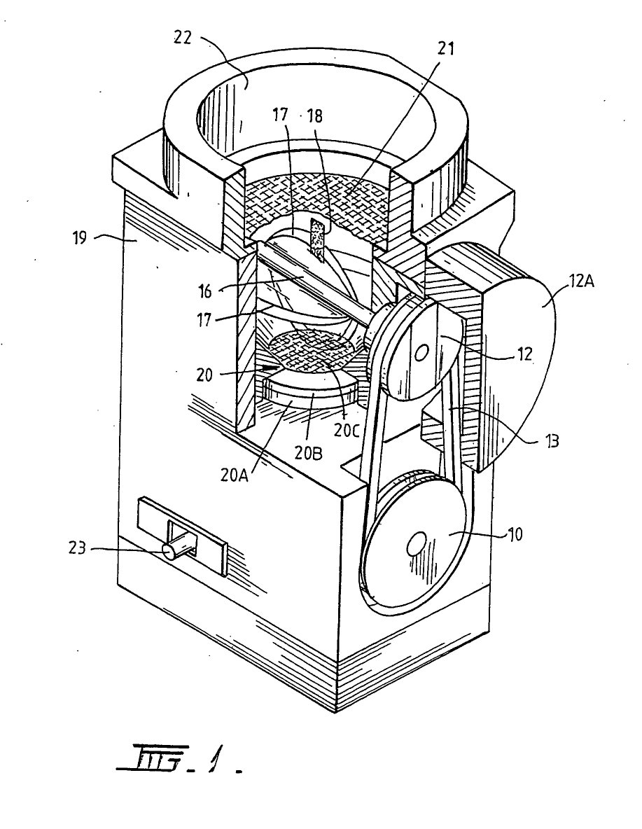

The apparatus shown in FIG. 1 includes a DC electromotor having a drive pulley 10 with a torque of at least 80 g-cm and operated using 2×1.5 V alkaline batteries size AA housed in a battery compartment 11. The motor pulley 10 drives a rotor pulley 12 of polymeric material through a silicon rubber drive belt 13 at speeds typically around 9000 rpm. The pulley 12 is mounted in an upper housing and covered by a cap 12A.

The assembly, consisting of motor, pulley and tension drive belt, is also an electrostatic friction generator, delivering up to 12,000 V DC to a rotor 15. The rotor 15 comprises a horizontal metal shaft 16 mounted for high speed rotation and a set of four equally spaced arcuate rotor blades 17 welded to the shaft. Each blade has a razor sharp outer edge with a central outwardly projecting flat silicon rubber tab 18. The rotor is mounted in a housing 19, at the bottom of which a support 20 is provided for receiving a quantity of micronised powder together with a quantity of beads. Typically 50 to 100 beads are provided each of a diameter around 2 mm, the beads being spherical and of silicon rubber. The support 20 comprises a stainless steel base 20A, a metallised porous substrate 20B, and a support mesh 20C.

At its upper end, the housing 20 mounts a discharge outlet screen 21 and above the mesh is situated a discharge port 22. The discharge outlet screen in a typical embodiment is a double mesh of nylon material, typically the upper mesh being of 10 micron material and the lower mesh being of 75 micron material, the two layers separated by a distance of between 0.1 and 1 mm. Fine alumina powder, for example, may be placed between the two layers of mesh to act as a filtering substance.

Use of this apparatus for inhalation therapy will now be described, this application being for the treatment of an asthmatic attack.

Pure micronised salbutamol powder (generic name: albuterol sulfate) is used as the therapeutic agent. 0.05 g of this powder was placed on the support 20 and the switch 22 closed to activate the device. It was found that discharge through the tube 22 comprised an effective solid aerosol which could readily and effectively treat patients.

Assessment of performance was conducted in a laboratory and on the basis of 1,000 individual experiments it was concluded that the output was highly respirable and there is strong probability that over 85% of the salbutamol base would enter the lower pulmonary system. The amount of delivered dose was very consistent, as expressed by the relative standard deviation (RSD) statistic, being regularly below 8.0%. The delivered dosage level was set to about 40 μg over 2-second runs.

For analysing these tests, the emission was analysed by capturing the 2-second output of the device by a standard Twin Impinger apparatus (USP 23/2) which separates the dust into respirable and non-respirable fractions. Subsequently, these were accurately determined by HPLC analysis, using a standard USP/CSIRO method for determining salbutamol sulphate fractions.

1. An apparatus for dispersing a fine powder, the apparatus including a container for receiving contents which include the fine powder and beads of a relatively large dimension compared with the powder, a rotor rotatably mounted within the container for co-dispersing the contents when the rotor is driven, the rotor being adapted to be rotated at a relative high speed and having means for establishing an electric field in the container and for mechanically dispensing the contents of the container, a discharge port from the container having means to control discharge of a dispersion of fine powder from within the container and to retain the beads within the container.

2. An apparatus in accordance with claim 1, wherein the electric field establishing means of the rotor comprises an electrostatic charge generator connected to an axial extending rotable shaft portion of the rotor and the rotor has a plurality of metal arms extending in respective planes extending axially and radially.

3. An apparatus in accordance with claim 2, wherein the rotor is adapted to be driven at around 9000 rpm by an electric motor, and the apparatus includes a drive belt and driven pulley for the rotor of polymeric material for generating electrostatic charges, the metal arms being connected to the rotatable shaft portion which is of metal.

4. An apparatus in accordance with claim 3, wherein each of the arms is blade-like with a sharp edge directed outwardly from the shaft portion.

5. An apparatus in accordance with claim 4, wherein the metal arms are accurate with a razor-sharp periphery over the majority of the edge, and the arms comprise four which are spaced uniformly around the axis of the rotor.

6. An apparatus in accordance with claim 5, wherein each blade has an outwardly extending projecting tab of resilient, insulating material for acting as a paddle to interact with contents of the container.

7. An apparatus in accordance with claim 1, wherein the discharge control means comprises an outer mesh layer for transmitting particles of up to about 10 microns and an inner mesh layer, spaced inwardly from the outer layer, and having a mesh sizal for transmitting particles of up to about 75 microns.

8. An apparatus in accordance with claim 7, wherein the outer and inner mesh layers are spaced apart by up to about 1 mm and the space contains a filtering fine powder.

9. An apparatus in accordance with claim 1, wherein the beads are around 2-3 mm in diameter and around 50-100 beads are included in the container.

10. An apparatus in accordance with claim 9, wherein the beads are of a resilient insulating and wear resistant material.

11. An apparatus in accordance with claim 1, and having a discharge duct connected to the discharge control means for inhalation therapy, the contents comprising a micronised powder pharmaceutical and millimetre sized polymeric beads, the apparatus further comprising electrical control means to create a short burst of a discharged cloud of pharmaceutical to provide a controlled dose.

12. An apparatus in accordance with claim 11, wherein the container incorporates the rotor and contents and is detachably mountable on a base unit which mounts batteries, the electrical control means and a motor to drive the rotor at around 9000 rpm.

13. A method of inhalation therapy comprising using the apparatus as claimed in claim 1 to periodically discharge on demand a short burst of micronised powder pharmaceutical.

14. An apparatus for dispersing a fine powder, the apparatus including a container for receiving contents which include the fine powder and beads of a relatively large dimension compared with the powder, a rotor rotatably mounted within the container for co-dispersing the contents when the rotor is driven, the rotor being adapted to be rotated at a relative high speed and having means for establishing an electric field in the container and for mechanically dispensing the contents of the container, a discharge port from the container having means to control discharge of a dispersion of fine powder from within the container and to retain the beads within the container, wherein the discharge control means comprises an outer mesh layer for transmitting particles of up to about 10 microns and an inner mesh layer, spaced inwardly from the outer layer, and having a mesh sizal for transmitting particles of up to about 75 microns, and wherein the outer and inner mesh layers are spaced apart by up to about 1 mm and the space contains a filtering fine powder.

15. An apparatus according to claim 14 and wherein the beads are around 2-3 mm in diameter and around 50-100 beads are included in the container.

16. An apparatus according to claim 15, wherein the beads are of a resilient insulating and wear resistant material.

17. An apparatus according to claim 15, and further comprising a discharge duct connected to the discharge control means for inhalation therapy, the contents comprising a micronised powder pharmaceutical and millimetre sized polymeric beads, the apparatus further comprising electrical control means to create a short burst of a discharged cloud of pharmaceutical to provide a controlled dose.

18. An apparatus according to claim 17, wherein the container incorporates the rotor and contents and is detachably mountable on a base unit which mounts batteries, the electrical control means and a motor to drive the rotor at around 9000 rpm.
